(19) United States
(12) Patent Application Publication
(10) Pub. No.: US 2026/0207489 A1
(43) Pub. Date: Jul. 23, 2026

(54) FINGOLIMOD ORAL LIQUID COMPOSITION AND USE THEREOF

(71) Applicant: SUZHOU HUAJIAN RUIDA PHARMACEUTICAL TECHNOLOGY CO., LTD, Shanghai (CN)

(72) Inventors: Jisheng GE, Suzhou, Jiangsu (CN); Xuemin GE, Suzhou, Jiangsu (CN); Shufang LIU, Suzhou, Jiangsu (CN); Taiming YU, Suzhou, Jiangsu (CN); Qinrong SANG, Suzhou, Jiangsu (CN); Lian WANG, Suzhou, Jiangsu (CN); Ye ZHOU, Suzhou, Jiangsu (CN)

(21) Appl. No.: 19/143,016

(22) PCT Filed: Jul. 10, 2024
(86) PCT No.: PCT/CN2024/104798

§ 371 Date: Jun. 25, 2025

(30) Foreign Application Priority Data
May 13, 2024 (CN) 202410590969.2

(51) Int. Cl. A61K 9/00; A61K 9/08; A61K 31/137; A61K 47/02; A61K 47/12; A61K 47/18; A61K 47/40

(52) U.S. Cl. CPC A61K 9/0095; A61K 9/08; A61K 31/137; A61K 47/02; A61K 47/12; A61K 47/183; A61K 47/40

(57) ABSTRACT

An oral liquid composition contains fingolimod or a pharmaceutically acceptable salt thereof. The oral liquid composition at least contains fingolimod or a pharmaceutically acceptable salt thereof, cyclodextrin, a metal complexing agent, and water. The mass ratio of the cyclodextrin to the fingolimod or the pharmaceutically acceptable salt thereof is 70-200:1. The composition has a pH of 3-6. By means of adding the metal complexing agent, providing the specific amount of cyclodextrin, and controlling the pH of the composition, the problems of physical and chemical instability of fingolimod in the oral liquid composition caused by of iron ion leaching in the production line, long-time storage, environmental change, etc. are solved.

The present disclosure claims the priority to patent application No. 202410590969.2 entitled “FINGOLIMOD ORAL LIQUID COMPOSITION AND USE THEREOF” and filed on May 13, 2024, the content of which is incorporated herein by reference in its entirety.

### TECHNICAL FIELD

The present disclosure relates to the field of biopharmaceuticals, and in particular, to a fingolimod oral liquid composition and use thereof.

### BACKGROUND

Fingolimod (CAS No. 162359-55-9; chemical name: 2-amino-2-[2-(4-octylphenyl)ethyl]-1,3-propanediol) is an SIP (sphingosine-1-phosphate) receptor modulator. It can prevent lymphocytes from leaving secondary lymphoid organs and prevent reactive lymphocytes from entering the central nervous system. In clinical applications, fingolimod is primarily used for preventing the recurrence of multiple sclerosis and the progression of body disability.

Various compositions containing fingolimod or its salt in oral solid forms, such as capsules and orally disintegrating tablets, are commercially available in the market, but there are no oral liquid formulations in the market. Therefore, there is a need for oral liquid compositions to improve patient compliance.

However, due to the requirement of long-term storage and varied storage or transport environments to facilitate distribution, the stability, especially the long-term stability of formulation, is pivotal in ensuring the quality of oral liquid compositions.

In actual pharmaceutical production lines, liquid compositions containing fingolimod or its salt are inevitably in contact with production line devices (e.g., stainless steel), which may, more or less, release iron ions. The inventors of the present disclosure surprisingly found that, since the alkylarene group of fingolimod is prone to being oxidized to form a ketone and the iron ions exhibit catalytic effects on oxidation of fingolimod, the stability of the fingolimod liquid composition will be affected by the iron ions, even in a trace amount.

### SUMMARY

In light of the fact, the present disclosure provides a fingolimod oral liquid composition with long-term stability and good weatherability as follows.

The present disclosure provides a fingolimod oral liquid composition, comprising the following components:
(A) 0.0001% to 0.5% of fingolimod or a pharmaceutically acceptable salt thereof;
(B) a cyclodextrin, wherein the mass ratio of the cyclodextrin to fingolimod or the pharmaceutically acceptable salt thereof is 70-200:1;
(C) 0.01% to 1% of a metal complexing agent; and
(D) water,
wherein the pH of the oral liquid composition is 3-6.

Preferably, in the oral liquid composition, the cyclodextrin is at least one of a natural cyclodextrin, a branched cyclodextrin, an alkyl cyclodextrin, and a hydroxyalkyl cyclodextrin.

In the present disclosure, the natural cyclodextrin is at least one of α-cyclodextrin, β-cyclodextrin, and γ-cyclodextrin.

In the present disclosure, the “branched cyclodextrin” refers to a natural cyclodextrin with a glycosyl branch on the ring (e.g., glucose, maltose, cellobiose, lactose, sucrose, galactose, glucosamine, and the like). The branched cyclodextrin is formed by bonding a glycosyl to the ring of a natural cyclodextrin with the help of a pullulanase. For example, the branched cyclodextrin can be obtained by bonding a glycosyl such as glucosyl to an oxygen atom at position 6 on 1, 2, 3, or more glucose residues in a natural cyclodextrin via α-1,6 bond.

In the present disclosure, the “alkyl cyclodextrin” refers to a natural cyclodextrin in which a hydrogen atom on hydroxyl is substituted with an alkyl. For example, the alkyl may be a C₁₋₈ alkyl. Specifically, the alkyl may be methyl, ethyl, propyl, butyl, pentyl, hexyl, heptyl, octyl, or the like.

In the present disclosure, the “hydroxyalkyl cyclodextrin” refers to a natural cyclodextrin in which a hydrogen atom on hydroxyl is substituted with a hydroxyalkyl. For example, the hydroxyalkyl may be a C₁₋₈ hydroxyalkyl. Specifically, the hydroxyalkyl may be hydroxymethyl, hydroxyethyl, hydroxypropyl, hydroxybutyl, hydroxypentyl, hydroxyhexyl, hydroxyheptyl, hydroxyoctyl, or the like, with 1-6 hydroxyl groups.

Preferably, in the fingolimod oral liquid composition, the content of fingolimod or the pharmaceutically acceptable salt thereof is 0.001% to 0.1%, preferably 0.005% to 0.05%.

Preferably, in the fingolimod oral liquid composition, the mass ratio of the cyclodextrin to fingolimod or the pharmaceutically acceptable salt thereof is 70-100:1.

Preferably, fingolimod or the pharmaceutically acceptable salt thereof is fingolimod hydrochloride or fingolimod dodecanesulfonate.

Preferably, fingolimod or the pharmaceutically acceptable salt thereof is fingolimod hydrochloride.

Preferably, in the oral liquid composition, the content of the metal complexing agent is 0.05% to 0.5%; preferably 0.1% to 0.2%.

Preferably, in the oral liquid composition, the cyclodextrin is hydroxypropyl-β-cyclodextrin.

Preferably, in the oral liquid composition, the metal complexing agent is at least one of disodium edetate and sodium calcium edetate, preferably disodium edetate.

Preferably, the oral liquid composition further comprises:
(E) a buffering agent, wherein the buffering agent is present in an amount sufficient to maintain the oral liquid composition at a pH of 3-6.

Preferably, in the oral liquid composition, the buffering agent is at least one of citric acid and a hydrate thereof, disodium hydrogen phosphate and a hydrate thereof, sodium citrate and a hydrate thereof, a phosphate and a hydrate thereof, malic acid, tartaric acid, hydrochloric acid, and sodium hydroxide. Preferably, the buffering agent is at least one of citric acid and a hydrate thereof, disodium hydrogen phosphate and a hydrate thereof, and sodium citrate and a hydrate thereof.

Preferably, in the oral liquid composition, the amount of the buffering agent is 0.03% to 3%.

Preferably, the oral liquid composition may further comprise an additional pharmaceutically acceptable excipient, wherein the additional pharmaceutically acceptable excipient is at least one of a preservative, a sweetener, and a flavoring agent.

Preferably, the amount of the additional pharmaceutically acceptable excipient is 0.01% to 20%, preferably 0.1% to 10%.

The present disclosure further provides use of the oral liquid composition described above for manufacturing a medicament or a formulation for treating an SIP-mediated disease, e.g., an immune disease, wherein preferably, the disease is multiple sclerosis.

### DETAILED DESCRIPTION

More details are provided in the descriptions hereinafter to help comprehend the present disclosure more thoroughly. However, it is apparent to those skilled in the art that the present disclosure can be implemented without one or more of those details. In other examples, in order to avoid conflation of the present disclosure, some technical features well known in the art are not described.

Unless specified, the “%” in the present disclosure refers to a mass-to-volume fraction in g/mL, and the “part” refers to a part by mass.

Hereinafter, the embodiments of the present disclosure are described in detail with reference to the following terminology:

### I. Oral Liquid Composition

The oral liquid composition of the present disclosure comprises the following components:
(A) 0.0001% to 0.5% of fingolimod or a pharmaceutically acceptable salt thereof;
(B) a cyclodextrin, wherein the mass ratio of the cyclodextrin to fingolimod or the pharmaceutically acceptable salt thereof is 70-200:1;
(C) 0.01% to 1% of a metal complexing agent; and
(D) water,
wherein the pH of the oral liquid composition is 3-6.

### (A) Active Pharmaceutical Ingredient

In some embodiments, the active pharmaceutical ingredient of the present disclosure comprises fingolimod or a pharmaceutically acceptable salt thereof.

In some embodiments, the active pharmaceutical ingredient of the present disclosure is fingolimod or a pharmaceutically acceptable salt thereof.

In some embodiments, the pharmaceutically acceptable salt of the present disclosure refers to a salt acceptable for administration to a patient prepared from a base or an acid. In the case of fingolimod, the salt may be derived from a pharmaceutically acceptable inorganic acid or organic acid. Specifically, the pharmaceutically acceptable inorganic acid or organic acid includes an inorganic acid, such as hydrochloric acid, sulfuric acid, phosphoric acid, pyrophosphoric acid, hydrobromic acid, hydroiodic acid, and nitric acid; and an organic acid, such as citric acid, fumaric acid, gluconic acid, glutamic acid, lactic acid, maleic acid, malic acid, mandelic acid, oxalic acid, pantothenic acid, succinic acid, tartaric acid, acetic acid, sulfonic acid, alkylsulfonic acid, naphthalenesulfonic acid, and the like. In the present disclosure, fingolimod is particularly preferably a salt derived from hydrochloric acid or dodecanesulfonic acid.

In the oral liquid composition, the content of fingolimod or the pharmaceutically acceptable salt thereof of the present disclosure is 0.0001% to 0.5%, 0.0005% to 0.2%, 0.001% to 0.1%, 0.002% to 0.09%, 0.005% to 0.08%, 0.006% to 0.05%, or 0.008% to 0.03%. For example, the content may be 0.005%, 0.006%, 0.007%, 0.008%, 0.009%, 0.01%, 0.011%, 0.012%, 0.013%, 0.015%, 0.02%, 0.025%, and the like.

### (B) Cyclodextrin

In some embodiments, the cyclodextrin of the present disclosure may be a natural cyclodextrin, a branched cyclodextrin, an alkyl cyclodextrin, or a hydroxyalkyl cyclodextrin, specifically, for example, α-cyclodextrin, β-cyclodextrin, γ-cyclodextrin, dodecakis-2,6-O-methyl-α-cyclodextrin, tetradecakis-2,6-O-methyl-β-cyclodextrin, hexadecakis-2,6-O-methyl-γ-cyclodextrin, tetradecakis-2,6-O-ethyl-β-cyclodextrin, an α-cyclodextrin partially etherified with 2-hydroxypropyl, a β-cyclodextrin partially etherified with 2-hydroxypropyl (HP-β-CD), or a branched α-cyclodextrin or branched β-cyclodextrin obtained by bonding glucose or maltose via an α-1,6-glycosidic bond, and the like.

In some embodiments, the cyclodextrin of the present disclosure is β-cyclodextrins.

In some embodiments, the cyclodextrin of the present disclosure is hydroxyalkyl-β-cyclodextrins, such as hydroxypropyl-β-cyclodextrin or β-cyclodextrin partially etherified with 2-hydroxypropyl.

In some embodiments, the mass ratio of the cyclodextrin to fingolimod or the pharmaceutically acceptable salt thereof of the present disclosure is 70-200:1, 70-100:1, or 70-80:1. For example, the content of the cyclodextrin may be 75, 80, 85, 90, 95, 100, 105, 110, 120, 150, 180, etc., times that of fingolimod or the pharmaceutically acceptable salt thereof. Only a proper content of the cyclodextrin may ensure the stability of the oral liquid composition of the present disclosure.

### (C) Metal Complexing Agent

The metal complexing agent of the present disclosure refers to a reagent capable of complexing with a metal ion to stabilize same.

In some embodiments, the metal complexing agent of the present disclosure is particularly capable of complexing with Fe³⁺.

In some embodiments, the metal complexing agent of the present disclosure is selected from sodium citrate, disodium edetate, sodium calcium edetate, gentisic acid ethanolamine, oxyquinoline sulfate, and the like. In some embodiments, the metal complexing agent of the present disclosure is disodium edetate.

In some embodiments, in the oral liquid composition, the content of the metal complexing agent of the present disclosure is 0.01% to 1%, 0.02% to 0.8%, 0.05% to 0.5%, 0.08% to 0.3%, or 0.1% to 0.2%.

### (D) Water

Water is the main solvent for the oral liquid composition of the present disclosure, and is present in an amount sufficient to ensure long-term weatherability and stability of all components in the composition.

In some embodiments, a single dose of the oral liquid composition of the present disclosure may has a volume of 0.1-500 mL, for example 0.2 mL, 0.5 mL, 1 mL, 2 mL, 5 mL, 10 mL, 20 mL, 50 mL, 100 mL, 120 mL, 150 mL, 200 mL, 250 mL, and the like. The composition is diluted to the designated volumes with water.

### (E) Buffering Agent

The pH of the oral liquid composition of the present disclosure is 3-6. For example, the pH may be 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, and the like.

In some embodiments, the oral liquid composition is adjusted to the aforementioned pH values by adding a pH buffering agent.

In some embodiments, the buffering agent is at least one of citric acid and a hydrate thereof, disodium hydrogen phosphate and a hydrate thereof, sodium citrate and a hydrate thereof, a phosphate and a hydrate thereof, malic acid, tartaric acid, hydrochloric acid, and sodium hydroxide.

In some embodiments, the buffering agent is at least one of citric acid and a hydrate thereof, disodium hydrogen phosphate and a hydrate thereof, and sodium citrate and a hydrate thereof.

In some embodiments, in the oral liquid composition, the amount of the buffering agent is 0.03% to 3%. For example, the amount may be 0.05%, 0.1%, 0.2%, 0.5%, 1%, 1.5%, 2%, 2.5%, or 2.8%.

### (F) Additional Pharmaceutically Acceptable Excipient

An additional pharmaceutically acceptable excipient may be added in accordance with the needs, provided that the oral liquid composition of the present disclosure is not affected.

In some embodiments, in the composition, the content of the excipient may be 0.01% to 20% or 0.1% to 10%.

In the present disclosure, the pharmaceutically acceptable excipient that may be added may comprise, for example, at least one of a preservative, a sweetener, and a flavoring agent.

In some embodiments, the preservative of the present disclosure is at least one of benzoic acid, sorbic acid, and sodium benzoate, preferably sodium benzoate. In some embodiments, in the composition, the content of the preservative may be 0.01% to 0.5%.

In some embodiments, in order to endow the composition with a better taste, a sweetener may be added. The sweetener may be, e.g., at least one of sorbitol, mannitol, sucralose, aspartame, and sodium saccharin, preferably at least one of mannitol or sucralose. In some embodiments, in the composition, the content of the sweetener may be 0.2% to 10%.

In some embodiments, in order to endow the composition with a better flavor, a flavoring agent may be added. The flavoring agent may be, e.g., at least one of a sweet orange flavor, a strawberry flavor, a tangerine flavor, a vanilla flavor, or a mix fruit flavor. In some embodiments, in the composition, the content of the flavoring agent may be 0% to 5%.

### II. Formulation

The pharmaceutical composition of the present disclosure may be formulated into an oral liquid formulation.

In some embodiments, a single dose of the oral liquid formulation of the present disclosure may has a volume of 0.1-500 mL, for example 0.2 mL, 0.5 mL, 1 mL, 2 mL, 5 mL, 10 mL, 20 mL, 50 mL, 100 mL, 120 mL, 150 mL, 200 mL, 250 mL, and the like. The composition is diluted to the designated volumes with water.

### III. Use

The pharmaceutical composition or formulation of the present disclosure may be used for treating a disease, e.g., an immune disease. In some embodiments, the disease is multiple sclerosis.

By means of adding a metal complexing agent and a specific amount of cyclodextrin to the fingolimod liquid composition and controlling the pH of the composition, the present disclosure solves the problem of instability of the oral liquid composition due to iron ion release in the production line, long-term storage, environmental changes in storage, etc, and provides a novel dosage form-oral liquid, which has good prospects in large-scale production for clinical use and improved patient compliance.

The pharmaceutical composition or formulation of the present disclosure has good long-term stability, including physical and/or chemical stability. Furthermore, the pharmaceutical composition or formulation of the present disclosure also has good weatherability, including resistance to high temperature and/or resistance to low temperature.

The term “chemical stability” refers to that related substances in the composition barely change after storing at a high temperature of 60° C. for at least 30 days.

The term “physical stability” refers to that no crystals precipitate from the composition after storing at a low temperature of 2-8° C. for at least 10 days.

The term “related substances” refer to impurities such as starting materials, intermediates, polymers, and by-products introduced in the pharmaceutical composition during the manufacturing process, degradation products during the storage process, and the like.

The term “reporting threshold” is the lowest limit of impurity to be reported, that is, an impurity is needed to be reported if its content is higher than the limit.

The present disclosure is further illustrated in detail by the following specific examples.

### Raw Materials

Fingolimod hydrochloride: having a purity of greater than 99.0%, a drug substance conforming to the requirements of the US Pharmacopeia.

Hydroxypropyl-β-cyclodextrin: having a molecular weight of 1431-1806, a pharmaceutically acceptable excipient conforming to the Chinese Pharmacopoeia.

Other reagents: purchased from Sinopharm Chemical Reagent Co., Ltd., for direct use.

### Assay Method Impurity Assay:

Instruments: high-performance liquid chromatograph, Agilent Technologies, Inc., Agilent 1260.
Procedures: The solvent was acetonitrile-0.1% phosphoric acid solution (50:50).
Preparation of test sample solution: An appropriate amount of sample was taken from each example, dissolved in the above solvent and quantitatively diluted with the solvent to obtain a solution containing about 0.8 mg of the sample per 1 mL.
Preparation of reference solution: an appropriate amount of the fingolimod hydrochloride reference with accurately calibrated content was quantitatively dissolved with the solvent to obtain a solution containing about 4 μg of fingolimod hydrochloride per 1 mL.
Preparation of the sensitivity solution: 1 mL of the reference solution was placed into a 10 mL volumetric flask, diluted to the volume with the solvent, and shaken uniformly.
System suitability solution: 6 mg of a mixed fingolimod hydrochloride reference standard (including fingolimod hydrochloride and various impurities; USP reference standard) was placed into a 10 mL volumetric flask, diluted to the volume with a diluent, and shaken dissolving uniformly.
Chromatographic conditions: octadecylsilane-bonded silica was used as the filler (Prontosil 120-3-C18 ACE-EPS (150 mm×3 mm, 3 μm) or an equivalent chromatographic column); 0.1% phosphoric acid was used as mobile phase A and acetonitrile was used as mobile phase B with the gradients shown in the following table; the column temperature was 40° C.; the detection wavelength was 215 nm; the flow rate was 0.8 mL/min; and the sample injection volume was 5 μL.

| Time (min) | Mobile phase A (%) | Mobile phase B (%) |
|---|---|---|
| 0 | 80 | 20 |
| 20 | 5 | 95 |
| 23 | 5 | 95 |
| 23.1 | 80 | 20 |
| 33 | 80 | 20 |

System suitability requirement: the chromatogram of the system suitability solution should have peak sequences of fingolimod hexyl homolog, fingolimod heptyl homolog, fingolimod, O-acetyl fingolimod, fingolimod nonyl homolog, fingolimod decyl homolog, 3-phenethyl homolog, and 2-phenethyl homolog, in which the resolution between the O-acetyl fingolimod peak and the fingolimod nonyl homolog peak should be not less than 1.2. In the chromatogram of the sensitivity solution, the signal-to-noise ratio of the main peak height should be not less than 10.
Determination procedure: the test sample solution and the reference solution were precisely measured and separately injected into the liquid chromatograph, and the chromatogram was recorded.

### Examples 1-5. Effects of pH of Composition

Fingolimod hydrochloride pharmaceutical compositions were prepared according to the formula shown in Table 1. The specific procedures were as follows: hydroxypropyl-β-cyclodextrin was added to a certain amount of purified water to give a solution; fingolimod hydrochloride was then dissolved in the solution; the other excipients were sequentially added; the mixture was stirred for dissolution; finally, purified water was added to dilute the solution to the volume. The compositions of the examples were sealed in ampoules and preserved in a high temperature condition of 60° C. for 30 days before the impurity content was determined.

TABLE 1

| Component (mg) | | | Example 1 | Example 2 | Example 3 | Example 4 | Example 5 |
|---|---|---|---|---|---|---|---|
| Fingolimod hydrochloride | | | 22.4 | 22.4 | 22.4 | 22.4 | 22.4 |
| Mannitol | | | 4800 | 4800 | 4800 | 4800 | 4800 |
| Hydroxypropyl-β-cyclodextrin | | | 1792 | 1792 | 1792 | 4480 | 4480 |
| Citric acid | | | 768 | 200 | 200 | — | — |
| Sodium citrate | | | 0 | 157 | — | — | — |
| Disodium hydrogen phosphate | | | — | — | 800 | 800 | 800 |
| Sodium dihydrogen phosphate | | | — | — | — | 200 | 233 |
| Sodium benzoate | | | 200 | 200 | 200 | 200 | 200 |
| Disodium edetate | | | 200 | 200 | 200 | 200 | 200 |
| Sucralose | | | 200 | 200 | 200 | 200 | 200 |
| Sodium hydroxide | | | — | — | — | — | q.s. |
| Volume added with purified water | | | 200 mL | 200 mL | 200 mL | 200 mL | 200 mL |
| pH | | | 3.0 | 4.5 | 6.0 | 7.0 | 8.0 |
| Related | Day 0 | Maximum | <0.05% | <0.05% | 0.05%** | 0.10% | 0.10% |
| substance* | | individual | | | | | |
| | | impurity | | | | | |
| | | Total | <0.05% | <0.05% | 0.05%** | 0.18% | 0.10% |
| | | impurity | | | | | |
| | 60° C. | Maximum | <0.05% | <0.05% | <0.05% | 0.11% | 0.12% |
| | Day 10 | individual | | | | | |
| | | impurity | | | | | |
| | | Total | <0.05% | <0.05% | <0.05% | 0.19% | 0.19% |
| | | impurity | | | | | |
| | 60° C. | Maximum | <0.05% | <0.05% | <0.05% | 0.22% | 0.58% |
| | Day 30 | individual | | | | | |
| | | impurity | | | | | |
| | | Total | <0.05% | <0.05% | <0.05% | 0.69% | 1.54% |
| | | impurity | | | | | |

*Reporting threshold: 0.05%.
**For the assay data of Example 3, the maximum individual impurity and the total impurity were 0.05% on day 0 and less than 0.05% on days 10 and 30, which were acceptable errors due to instrument precision and the like.

It can be seen, pH had a great effect on the chemical stability of the fingolimod hydrochloride oral liquid of the present disclosure, and the pharmaceutical composition exhibited good high-temperature stability at pH 3-6.

### Examples 6-10

Fingolimod hydrochloride pharmaceutical compositions were prepared according to the formula shown in Table 2. The specific procedures were the same as in Example 1. The obtained pharmaceutical compositions of the examples were sealed in glass bottles and refrigerated at 2-8° C. in a refrigerator for 5 days or 10 days. After the refrigeration, the samples were placed perpendicularly under a canopy light with an illumination of 1000 lx in a dark room and inspected, in a horizontal direction, if crystals were precipitated.

TABLE 2
Effects of amount of hydroxypropyl-β-cyclodextrin

| Component (mg) | Example 6 | Example 7 | Example 8 | Example 9 | Example 10 |
|---|---|---|---|---|---|
| Fingolimod hydrochloride | 22.61 | 22.58 | 22.58 | 22.4 | 22.4 |
| Mannitol | 4800 | 4800 | 4800 | 4800 | 4800 |
| Hydroxypropyl-β-cyclodextrin | 896 | 1120 | 1344 | 1568 | 1792 |
| Citric acid | 200 | 200 | 200 | 200 | 200 |
| Sodium citrate | 0 | 0 | 0 | 0 | 0 |
| Disodium hydrogen phosphate | 820 | 800 | 800 | 800 | 800 |
| Disodium edetate | 200 | 200 | 200 | 200 | 200 |
| Sodium benzoate | 200 | 200 | 200 | 200 | 200 |
| Sucralose | 200 | 200 | 200 | 200 | 200 |
| Volume added with purified | 200 mL | 200 mL | 200 mL | 200 mL | 200 mL |
| water | | | | | |
| pH | 6 | 6 | 6 | 6 | 6 |
| Hydroxypropyl-β- | 40:1 | 50:1 | 60:1 | 70:1 | 80:1 |
| cyclodextrin:fingolimod | | | | | |
| hydrochloride | | | | | |
| 2-8° C. refrigeration for | Precipitates | Precipitates | No | No | No |
| 5 days | were | were | precipitates | precipitates | precipitates |
| | observed | observed | were observed | were observed | were observed |
| | after | after | after | after | after |
| | refrigeration | refrigeration | refrigeration | refrigeration | refrigeration |
| 2-8° C. refrigeration for | Precipitates | Precipitates | Precipitates | No | No |
| 10 days | were | were | were | precipitates | precipitates |
| | observed | observed | observed | were observed | were observed |
| | after | after | after | after | after |
| | refrigeration | refrigeration | refrigeration | refrigeration | refrigeration |

It can be seen, the weight ratio of hydroxypropyl-β-cyclodextrin to fingolimod hydrochloride had a great effect on the physical stability of the pharmaceutical composition of the present disclosure. The pharmaceutical composition of the present disclosure exhibited good physical stability when the aforementioned ratio was 70 or greater.

### Examples 11-14. Effects of Complexing Agents

To simulate the production line environment of the pharmaceutical composition, a trace amount of Fe³⁺ was added to the formula.

Fingolimod hydrochloride pharmaceutical compositions were prepared according to the formula shown in Table 3. The specific procedures were the same as in Example 1. A specific amount of FeCl₃ was added before the solution was diluted to the volume. The obtained pharmaceutical compositions of the examples were sealed in glass ampoules and preserved under an illumination condition (the illumination was 4500 lx±500 lx; the total illumination of light source should be not lower than 1.2×10⁶ lux·hr; the energy of the near ultraviolet lamp should be not less than 200 W·hr/m²) for 10 days, before the related substances were determined.

| | Example | Example | Example | Example |
|---|---|---|---|---|
| Component (mg) | 11 | 12 | 13 | 14 |

| Fingolimod hydrochloride | 22.4 | | 22.4 | | 22.4 | | 22.4 | |
|---|---|---|---|---|---|---|---|---|
| Mannitol | 4800 | | 4800 | | 4800 | | 4800 | |
| Hydroxypropyl-β-cyclodextrin | 1792 | | 1792 | | 1792 | | 1792 | |
| Citric acid | 200 | | 200 | | 200 | | 200 | |
| Sodium citrate | 157 | | 157 | | 157 | | 157 | |
| Sodium benzoate | 200 | | 200 | | 200 | | 200 | |
| Disodium edetate | — | | — | | 200 | | 400 | |
| Sucralose | 200 | | 200 | | 200 | | 200 | |
| Volume added with purified water | 200 | mL | 200 | mL | 200 | mL | 200 | mL |
| Addition amount of Fe³⁺ | 1 | ppm | 5 | ppm | 1 | ppm | 1 | ppm |

| pH | | | 4.5 | 4.5 | 4.5 | 4.5 |
|---|---|---|---|---|---|---|
| Related | Day 0 | Maximum individual | <0.05% | 0.17% | <0.05% | <0.05% |
| substance | | impurity | | | | |
| | | Total impurity | <0.05% | 0.43% | <0.05% | <0.05% |
| | Illumination | Maximum individual | 0.11% | 0.26% | <0.05% | <0.05% |
| | Day 5 | impurity | | | | |
| | | Total impurity | 0.52% | 1.20% | <0.05% | <0.05% |
| | Illumination | Maximum individual | 0.14% | 0.27% | <0.05% | <0.05% |
| | Day 10 | impurity | | | | |

| | | | | |
|---|---|---|---|---|
| Total impurity | 0.69% | 1.24% | <0.05% | <0.05% |

It can be seen, the stability of the pharmaceutical composition of the present disclosure was susceptible to the effects of the iron ion release in the production line, and the chemical stability of the pharmaceutical composition can be improved by adding a complexing agent.

With reference to the aforementioned examples, all technical solutions within the principle of the present disclosure fall within the claimed scope of the present disclosure. It should be noted that certain improvements and embellishments can be made by those of ordinary skill in the art without departing from the principle of the present disclosure, and should also be construed as within the claimed scope of the present disclosure.

1. An oral liquid composition, comprising the following components:
(A) 0.0001% to 0.5% of fingolimod or a pharmaceutically acceptable salt thereof;
(B) a cyclodextrin, wherein the mass ratio of the cyclodextrin to fingolimod or the pharmaceutically acceptable salt thereof is 70-200:1;
(C) 0.01% to 1% of a metal complexing agent; and
(D) water,
wherein the pH of the oral liquid composition is 3-6.

2. The oral liquid composition according to claim 1, wherein the cyclodextrin is at least one of a natural cyclodextrin, a branched cyclodextrin, an alkyl cyclodextrin, and a hydroxyalkyl cyclodextrin.

3. The oral liquid composition according to claim 1, wherein:
the content of fingolimod or the pharmaceutically acceptable salt thereof is 0.001% to 0.1%, preferably 0.005% to 0.05%;
preferably, the mass ratio of the cyclodextrin to fingolimod or the pharmaceutically acceptable salt thereof is 70-100:1;
preferably, fingolimod or the pharmaceutically acceptable salt thereof is fingolimod hydrochloride or fingolimod dodecanesulfonate;
preferably, fingolimod or the pharmaceutically acceptable salt thereof is fingolimod hydrochloride;
preferably, the content of the metal complexing agent is 0.05% to 0.5%, preferably 0.1% to 0.2%.

4. The oral liquid composition according to claim 1, wherein the cyclodextrin is hydroxypropyl-β-cyclodextrin; preferably, the metal complexing agent is at least one of disodium edetate and sodium calcium edetate, preferably disodium edetate.

5. The oral liquid composition according to claim 1, further comprising:
(E) a buffering agent, wherein the buffering agent is present in an amount sufficient to maintain the oral liquid composition at a pH of 3-6.

6. The oral liquid composition according to claim 5, wherein the buffering agent is at least one of citric acid and a hydrate thereof, disodium hydrogen phosphate and a hydrate thereof, sodium citrate and a hydrate thereof, a phosphate and a hydrate thereof, malic acid, tartaric acid, hydrochloric acid, and sodium hydroxide; preferably, the buffering agent is at least one of citric acid and a hydrate thereof, disodium hydrogen phosphate and a hydrate thereof, and sodium citrate and a hydrate thereof.

7. The oral liquid composition according to claim 6, wherein the amount of the buffering agent is 0.03% to 3%.

8. The oral liquid composition according to claim 1, further comprising an additional pharmaceutically acceptable excipient, wherein the additional pharmaceutically acceptable excipient is at least one of a preservative, a sweetener, and a flavoring agent.

9. The oral liquid composition according to claim 8, wherein the amount of the additional pharmaceutically acceptable excipient is 0.01% to 20%, preferably 0.1% to 10%.

10. A method for treating an SIP-mediated disease, e.g., an immune disease, comprising administering a therapeutically effective amount of the oral liquid composition according to claim 1 to a subject in need thereof, wherein preferably, the disease is multiple sclerosis.
